(19) United States
(12) Patent Application Publication
(10) Pub. No.: US 2015/0094364 A1
(43) Pub. Date: Apr. 2, 2015

(54) METHOD OF USING THEAFLAVIN

(71) Applicant: Dongning Li, (US)

(72) Inventors: Shimin Li, Glastonbury, CT (US); Xueyin Ren, Jiangyin City (CN); Jianhong Liu, Jiangyin City (CN)

(21) Appl. No.: 14/502,645

(22) Filed: Sep. 30, 2014

Related U.S. Application Data
(60) Provisional application No. 61/885,247, filed on Oct. 1, 2013.

(51) Int. Cl. A61K 31/352; A61K 36/82

(52) U.S. Cl. CPC A61K 31/352; A61K 36/82
USPC 514/456

(57) ABSTRACT

A method for treating alcoholic liver diseases or for preventing or ameliorating at least one of the symptoms of an alcoholic liver disease, the method includes administering to a subject in need thereof an effective amount of theaflavin, an analog thereof, or a prodrug thereof, or includes administering to the subject a composition comprising an extract of tea and a physiologically acceptable carrier.

### CROSS REFERENCE TO RELATED APPLICATIONS

This application claims the benefit of U.S. Provisional Application No. 61/885,247 filed on Oct. 1, 2013. The contents of both applications are hereby incorporated by reference in their entirety.

### FIELD OF THE INVENTION

This disclosure relates to the field of treating and preventing alcoholic liver disease, and use of theaflavin or a theaflavin analog to treat and prevent alcoholic liver disease, and the use of a composition having a theaflavin-containing tea extract to treat and prevent the same.

### BACKGROUND OF THE INVENTION

An alcoholic liver disease (ALD) occurs after years of heavy drinking. Alcohol can cause inflammation in the liver. Over time, scarring and cirrhosis can occur as a result of alcohol overconsumption, leading to an alcoholic liver disease that includes fatty liver (steatosis), alcoholic hepatitis, and chronic hepatitis with hepatic fibrosis or cirrhosis. It is a major cause of liver disease in Western countries. Clinical symptoms include pain and swelling in the abdomen, decreased appetite and weight loss, nausea, vomiting, and fatigue.

Steatosis, or fatty liver, is the accumulation of fatty acids in liver cells. These can be seen as fatty globules under the microscope. Alcoholism causes development of large fatty globules (macro vesicular steatosis) throughout the liver and can begin to occur after a few days of heavy drinking. Alcohol is metabolized in liver, a process that generates NADH. A higher NADH concentration induces fatty acid synthesis while a decreased NAD level results in decreased fatty acid oxidation. Subsequently, the higher levels of fatty acids signal the liver cells to compound it to glycerol to form triglycerides. These triglycerides accumulate, resulting in fatty liver.

Alcoholic hepatitis is characterized by the inflammation of hepatocytes. According to a 1993 report of National Institute on Alcohol Abuse and Alcoholism (NIAAA), between 10% and 35% of heavy drinkers develop alcoholic hepatitis, which can occur concomitantly or in succession. While development of hepatitis is not directly related to the dose of alcohol, some people seem more prone to this reaction than others. The inflammation appears to predispose to liver fibrosis. Inflammatory cytokines (TNF-α, IL6 and IL8) are thought to be essential in the initiation and perpetuation of liver injury by inducing apoptosis and necrosis. One possible mechanism for the increased activity of TNF-α is the increased intestinal permeability due to liver disease. This facilitates the absorption of the gut-produced endotoxin into the portal circulation. The Kupffer cells of the liver then phagocytose endotoxin, stimulating the release of TNF-α. TNF-α then triggers apoptotic pathways through the activation of caspases, resulting in cell death.

Cirrhosis is a late stage of serious liver disease marked by inflammation (swelling), fibrosis (cellular hardening) and damaged membranes preventing detoxification of chemicals in the body, ending in scarring and necrosis (cell death). Between 10% to 20% of heavy drinkers will develop cirrhosis of the liver (NIAAA, 1993). Acetaldehyde may be responsible for alcohol-induced fibrosis by stimulating collagen deposition by hepatic stellate cells. The production of oxidants derived from NADPH oxi-dase and/or cytochrome P-450 2E1 and the formation of acetaldehyde-protein adducts damage the cell membrane. Symptoms include jaundice (yellowing), liver enlargement, and pain and tenderness from the structural changes in damaged liver architecture. Without total abstinence from alcohol use, will eventually lead to liver failure. Late complications of cirrhosis or liver failure include portal hypertension (high blood pressure in the portal vein due to the increased flow resistance through the damaged liver), coagulation disorders (due to impaired production of coagulation factors), ascites (heavy abdominal swelling due to build up of fluids in the tissues) and other complications, including hepatic encephalopathy and the hepatorenal syndrome.

Although steatosis (i.e., fatty liver) will develop in any individual who consumes a large quantity of alcoholic beverages over a long period of time, this process is transient and reversible. Hepatitis with abstinence can be reversible. However, the later stages of fibrosis and cirrhosis tend to be irreversible.

The incidence of mortality from ALDs is high. It is consistently one of the top ten leading causes of death in U.S. urban males. The therapeutic options for ALDs have been very limited and are mostly palliative and symptomatic.

Abstinence is the most important therapeutic intervention for patients with an ALD, as it improves the outcome of hepatic injury, decreases progression to cirrhosis, and improves survival at all stages of an ALD. Other effective treatments include drastic measures, e.g., liver transplantation.

As there are few good options of treating an ALD, there is an urgent need to develop new therapeutic strategies, especially an effective treatment without severe side effects for patients suffering from an ALD.

### SUMMARY OF THE INVENTION

This invention provides a method for treating an ALD or for preventing or ameliorating at least one of the symptoms of an ALD.

One aspect of this invention relates to a method that includes administering to a subject in need thereof an effective amount of theaflavin, an analog thereof, or a prodrug thereof.

The theaflavin contains theaflavin, theaflavin-3-gallate, theaflavin-3′-gallate, theaflavin 3,3′-digallate, or a combination thereof.

The theaflavin, an analog thereof, or a prodrug thereof can be administered to the subject in a dosage equivalent to 5-500 mg of theaflavin per kg of body weight per day. This administration can be performed orally, buccally, rectally, parenterally, intravenously, intraperitoneally, intradermal, subcutaneously, intramuscularly, trans-dermally, or intratracheally to the subject.

Examples of the alcoholic liver disease include steatosis, alcoholic hepatitis, and alcoholic liver cirrhosis.

The method of this invention can further include administering to the subject a second therapeutic agent.

Another aspect of this invention relates to a method for treating an ALD that includes administering to a subject in need thereof a composition comprising an extract of tea and a physiologically acceptable carrier.

The extract of tea has theaflavin which contains theaflavin, theaflavin-3-gallate, theaflavin-3′-gallate, theaflavin 3,3′-digallate, or a combination thereof.

The extract of tea can be administered to the subject in a dosage equivalent to 5-500 mg of theaflavin per kg of body weight per day.

The composition is a pharmaceutical composition that can be in a solid, semi-solid, liquid, or gaseous form.

Examples of the alcoholic liver disease are enumerated above.

Yet another aspect of this invention relates to a method for prevention or amelioration of at least one of the symptoms of alcoholic liver diseases. The method includes administering to a subject in need thereof a composition comprising an extract of tea and a physiologically acceptable carrier.

Examples of the extract of tea, the composition, and the alcoholic liver disease are enumerated above.

The details of the invention are set forth in the description below. Other features, objects, and advantages of the invention will be apparent from the description and from the claims.

### DETAILED DESCRIPTION

The present invention provides a method for treating alcoholic liver disease that comprises administering to a subject in need thereof an effective amount of theaflavin, an analog thereof, or a prodrug thereof.

The term alcoholic liver disease includes steatosis, alcoholic hepatitis, and alcoholic liver cirrhosis.

Theaflavins are a class of benzoannulenone compounds which are formed from oxidation reactions of polyphenolic compounds. There are 12 components in theaflavins, including theaflavin (TF), theaflavin-3-gallate (TFMG), theaflavin-3,3′-digallate (TFdiG) and theaflavin-3′-gallate (TFM′G), which are the four major components. Pure theaflavins are orange colors, form needle crystals, have melting points of 237-240° C., are soluble in water, methanol, ethanol, acetone, n-butanol and ethyl acetate, are slightly soluble in ethyl ether, and are insoluble in chloroform and benzene. Theaflavins in solution are clear orange in color and are slightly acidic with a pH value of about 5.7. The solution color is not affected by the pH of the tea extraction solution, but theaflavins are auto-oxidative in basic solution. The oxidation process increases with the pH value.

Thearubigins are a class of complex, inhomogeneous brown colored phenolic compounds, with a range of molecular weight of 1,000-40×10³. Due to inhomogeneity, unclear structure, and unknown properties, it is difficult to isolate and purify the thearubigins.

Tea polyphenols, including catechins and theaflavins, are known for reducing triglyceride, removing free radicals, having anti-oxidant, anti-bacteria, anti-virus, anti-tumor, anti-mutagenic, and odor removal properties, and treating cardiovascular diseases, etc. They are applied in pharmaceutical, nutraceutical and food additive fields.

One example of the method is that administration of theaflavin or a theaflavin analog can be in monotherapy or in combination therapy. The combination therapy comprises administering to a subject in need thereof an effective amount of theaflavin or a theaflavin analog in combination with a second therapeutic agent.

Another aspect of the invention is a method for treating alcoholic liver disease that comprises administering to a subject in need thereof a composition comprising a physiologically acceptable carrier and an extract of tea.

The term of extract of tea includes a mixture of theaflavin, theaflavin-3-gallate, theaflavin-3′-gallate and theaflavin 3,3′-digallate.

The composition includes a pharmaceutical composition. Forms of the composition include, but are not limited to, a solid, semi-solid, liquid or gaseous form.

An example of the method is wherein administration of the composition can be by orally, buccally, rectally, parenterally, intravenously, intraperitoneally, intradermal, subcutaneously, intramuscularly, trans-dermally, or intratracheally.

An example of the method is wherein the composition is orally administered to the subject.

Another example of the method is wherein the composition is intravenously administered to the subject.

An example of the method is wherein theaflavin is administered in a dosage of from about 5 mg to about 500 mg per kg of body weight.

Yet another aspect of the invention is a method for prevention or amelioration of at least one of the symptoms of ALDs that comprises administering to a subject in need thereof a composition comprising an extract of tea and a physiologically acceptable carrier.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated by reference in their entirety.

### EXAMPLE 1

### Use of Theaflavin to Treat an ALD in an Animal Model

To explore therapeutic effects of theaflavin (prepared according to the method described in U.S. Pat. No. 8,282,970) on ALDs, the following experiment was carried out.

6-week old male rats (Wistar rats) were stabilized and then divided into 5 groups with 10 rats per group (n=10). Rats overdosed with ethanol were used as an experimental model for human ALD. Four groups of rats were fed with ethanol at a dose of 6000 mg/kg BW (50% ethanol at 12 ml/kg BW) per day for 30 days. These four groups were divided into positive control, theaflavin (low), theaflavin (mid), and theaflavin (high). Theaflavin (capsule, Xiayie) was also fed to rat at a dose of 83 mg/kg BW (low), 250 mg/kg (mid), and 500 mg/kg (high). In addition, Fifth group was fed with water as a negative control. Animals were sacrificed after the 30-day study and livers were collected for further analyses.

Three liver markers, i.e., malondialdehyde (MDA, a marker of oxidative stress), glutathione (GSH), and triglyceride (TG) were measured. The positive control group showed elevated MDA (p<0.05) and TG (p<0.01), and reduced GSH, indicating the success for the ALD model. Theaflavin (mid) and theaflavin (high) groups showed lower MDA and TG when compared to positive controls. See Table 2.

Liver histopathology analysis also showed that heaflavin (mid) and theaflavin (high) groups had different readings in both liver cell fat rating and fat color rating compared to those of the positive control. See Tables 3 and 4.

TABLE 1
Effect of theaflavin on subject body weight, liver weight and liver/body ratio

| | Dose | | BW (beginning) | BW (intermediate) | BW (ending) | Liver Weight | Liver body ratio |
|---|---|---|---|---|---|---|---|
| Group | (mg/kg, BW) | n (rat) | (g) | (g) | (g) | (g) | (%) |
| Negative Control | 0 | 10 | 189.8 ± 6.18 | 274.2 ± 22.66 | 356.5 ± 32.14 | 9.09 ± 0.92 | 2.55 ± 0.13 |
| Positive Control | 0 | 10 | 193.7 ± 8.82 | 265.8 ± 15.39 | 331.8 ± 38.68 | 9.96 ±1.06 | 3.01 ± 0.12 |
| Theaflavin (Low) | 83 | 10 | 193.3 ± 8.06 | 256.1 ± 21.23 | 329.1 ± 19.17 | 9.19 ± 0.88 | 2.79 ± 0.22 |
| Theaflavin (Mid) | 250 | 10 | 192.3 ± 12.34 | 266.3 ± 17.10 | 342.9 ± 21.99 | 9.83 ± 1.12 | 2.86 ± 0.23 |
| Theaflavin (High) | 500 | 10 | 194.3 ± 9.86 | 272.3 ± 17.27 | 365.2 ± 44.39 | 10.34 ± 1.32 | 2.84 ± 0.27 |

TABLE 2
Effect of theaflavin on MDA, GSH and TG of the rat livers

| | Dose | | MDA | GSH | TG |
|---|---|---|---|---|---|
| Group | (mg/kg, BW) | n (rat) | (nmol/mg) | (mg/g) | (mmol/L) |
| Negative Control | 0 | 10 | 1.20 ± 0.19 | 15.35 ± 1.67 | 0.39 ± 0.27 |
| Positive Control | 0 | 10 | 1.52 ± 0.21** | 13.10 ± 2.40* | 1.01 ± 0.32** |
| Theaflavin (Low) | 83 | 10 | 1.32 ± 0.13 | 13.86 ± 1.80 | 0.62 ± 0.21* |
| Theaflavin (Mid) | 250 | 10 | 1.24 ± 0.49* | 13.91 ± 1.66 | 0.65 ± 0.14* |
| Theaflavin (High) | 500 | 10 | 1.23 ± 0.08** | 14.43 ± 0.69 | 0.64 ± 0.13* |

TABLE 3
Effect of theaflavin on histopathology of the rat livers

| | Liver cell fat rating | | | | |
|---|---|---|---|---|---|
| | Negative | Positive | Theaflavin | Theaflavin | Theaflavin |
| animal ID | control | control | (low) | (mid) | (high) |
| 1 | 0 | 3 | 2 | 2 | 1 |
| 2 | 0 | 2 | 2 | 1 | 1 |
| 3 | 0 | 1 | 1 | 2 | 2 |
| 4 | 0 | 2 | 1 | 1 | 1 |
| 5 | 1 | 3 | 3 | 1 | 1 |
| 6 | 0 | 3 | 1 | 0 | 1 |
| 7 | 0 | 3 | 2 | 2 | 0 |
| 8 | 0 | 1 | 1 | 2 | 3 |
| 9 | 0 | 1 | 1 | 1 | 1 |
| 10 | 0 | 2 | 2 | 2 | 2 |
| sum | 1 | 21 | 16 | 14 | 13 |

TABLE 4
Effect of theaflavin on histopathology of the rat livers

| | Dose | | |
|---|---|---|---|
| Group | (mg/kg, BW) | n (rat) | Fat color rating |
| Negative Control | 0 | 10 | 0.10 ± 0.32 |
| Positive Control | 0 | 10 | 2.10 ± 0.88** |
| Theaflavin (Low) | 83 | 10 | 1.60 ± 0.70 |
| Theaflavin (Mid) | 250 | 10 | 1.40 ± 0.70* |
| Theaflavin (High) | 500 | 10 | 1.30 ± 0.82* |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for treating an alcoholic liver disease or for preventing or ameliorating at least one of the symptoms of an alcoholic liver disease, the method comprising administering to a subject in need thereof an effective amount of theaflavin, an analog thereof, or a prodrug thereof.

2. The method according to claim 1, wherein the theaflavin comprises theaflavin, theaflavin-3-gallate, theaflavin-3′-gallate, theaflavin 3,3′-digallate, or a combination thereof.

3. The method according to claim 2, wherein the theaflavin, an analog thereof, or a prodrug thereof is administered to the subject in a dosage equivalent to 5-500 mg of theaflavin per kg of body weight per day.

4. The method according to claim 3, wherein the theaflavin, an analog thereof, or a prodrug thereof is administered orally, buccally, rectally, parenterally, intravenously, intraperitoneally, intradermal, subcutaneously, intramuscularly, trans-dermally, or intratracheally to the subject.

5. The method according to claim 1, wherein the alcoholic liver disease is steatosis, alcoholic hepatitis, or alcoholic liver cirrhosis.

6. The method according to claim 1, further comprising administering to the subject a second therapeutic agent.

7. A method for treating an alcoholic liver disease, the method comprising administering to a subject in need thereof a composition comprising an extract of tea and a physiologically acceptable carrier.

8. The method according to claim 7, wherein the extract of tea comprises theaflavin.

9. The method according to claim 8, the theaflavin comprises theaflavin, theaflavin-3-gallate, theaflavin-3′-gallate, theaflavin 3,3′-digallate, or a combination thereof.

10. The method according to claim 8, wherein the extract of tea is administered to the subject in a dosage equivalent to 5-500 mg of theaflavin per kg of body weight per day

11. The method according to claim 7, wherein the alcoholic liver disease is steatosis, alcoholic hepatitis, or alcoholic liver cirrhosis.

12. The method according to claim 7, wherein the composition is a pharmaceutical composition.

13. The method according to claim 12, wherein the composition is in a solid, semi-solid, liquid, or gaseous form.

14. A method for prevention or amelioration of at least one of the symptoms of an alcoholic liver diseases, the method comprising administering to a subject in need thereof a composition comprising an extract of tea and a physiologically acceptable carrier.

15. The method according to claim 14, wherein the extract of tea comprises theaflavin.

16. The method according to claim 15, wherein the theaflavin comprises theaflavin, theaflavin-3-gallate, theaflavin-3′-gallate, theaflavin 3,3′-digallate, or a combination thereof.

17. The method according to claim 15, wherein the extract of tea is administered to the subject in a dosage equivalent to 5-500 mg of theaflavin per kg of body weight per day

18. The method according to claim 14, wherein the alcoholic liver disease is steatosis, alcoholic hepatitis, or alcoholic liver cirrhosis.

19. The method according to claim 14, wherein the composition is a pharmaceutical composition.

20. The method according to claim 19, wherein the composition is in a solid, semi-solid, liquid, or gaseous form.
